(19) United States
(12) Patent Application Publication
(10) Pub. No.: US 2008/0221350 A1
(43) Pub. Date: Sep. 11, 2008

(54) Process for Preparing 2-Arylcarbonyl Compounds, 2-Aryl Esters and 2-Arylnitriles and their Heteroaromatic Analogues

(76) Inventors: Andreas Meudt, Hofheim (DE); Sven Nerdinger, Kiefersfelden (DE); Bernd Wilhelm Lehnemann, Frankfurt am Main (DE); Till Vogel, Mannheim (DE); Victor Snieckus, Kingston (CA)

Correspondence Address: PROPAT, L.L.C., 425-C SOUTH SHARON AMITY ROAD, CHARLOTTE, NC, 28211-2841, US

(21) Appl. No.: 12/064,482

(22) PCT Filed: Sep. 12, 2006
(86) PCT No.: PCT/EP2006/008862

§ 371 Date: Feb. 22, 2008

(30) Foreign Application Priority Data
Sep. 22, 2005 (DE) 10 2005 045 132.2

(51) Int. Cl. C07C 253/30; C07C 45/61; C07C 67/28

(52) U.S. Cl. 558/308; 568/323; 560/8

(57) ABSTRACT

Process for preparing compounds by cross-coupling of enolizable carbonyl compounds, nitriles or their analogues with substituted aryl or heteroaryl compounds in the presence of a Brönsted base and of a catalyst or precatalyst containing a.) a transition metal, a complex, a salt or a compound of this transition metal from the group V, Mn, Fe, Co, Ni, Rh, Pd, Ir, Pt) and b.) at least one sulphonated phosphane ligand in a solvent or solvent mixture.

2-Aryl- or -heteroaryl-substituted carbonyl compounds and nitrites are a frequent structural motif in natural substances, physiologically active compounds and chemical precursors. However, their significance in modern organic synthesis is restricted by limitations in the availability of these compound classes, in particular when further functionalities are present in the target structure. More particularly, the selective bonding of functionalized aromatics or heteroaromatics to complex carbonyl compounds and their analogues still presents difficulties, since the standard processes for 2-functionalization of carbonyl compounds and their analogues—the reaction of their enols or enolates with electrophiles—is applicable to haloaromatics or—heteroaromatics only in exceptional cases, specifically when strongly electron-withdrawing substituents which promote nucleophilic aromatic substitution are present (see, for example, March, Advanced Organic Chemistry, Ch. 13: Aromatic Nucleophilic Substitution, p. 641-676). Moreover, the harsh reaction conditions needed are generally incompatible with sensitive functionalities.

More recent developments avoid these difficulties by accomplishing the linkage of enolates to aryl or heteroaryl halides with the aid of Pd or Ni catalysts in the presence of various ligands which prevent the otherwise dominant reductive elimination (Culkin, Hartwig, Acc. Chem. Res. 2003, 36, 235-245). However, the currently known processes all still have process technology or economic disadvantages which considerably restrict the scope of use. Among these, mention should be made here of the high costs of the catalysts/ligands, high required loadings/catalyst concentrations and difficult removability of the catalyst from the end product. One reason for the latter is also that the ligands used to date are all substantially nonpolar and, as a result, reside preferentially in the organic phase together with the metal in aqueous workups.

It would be very desirable to have a process which can convert substituted carbonyl compounds or nitrites with haloaromatics or haloheteroaromatics to the corresponding 2-aryl- or 2-heteroaryl-substituted carbonyl or nitrile compounds, simultaneously achieves very high yields, needs only very small amounts of catalyst and additionally features easy removal of the ligand and of the transition metal used from the product. As already mentioned, the synthesis processes published for this purpose to date do not satisfactorily solve this problem, as will be demonstrated further with reference to a few examples:
Use of expensive ligands (e.g. P^{t}Bu₃, Hartwig et al., U.S. Pat. No. 6,072,073) and complicated isolation of the product by chromatography
Use of ligands which are difficult to synthesize (ferrocene-based ligands, Hartwig et al., U.S. Pat. No. 6,057,456), complicated isolation of the product by chromatography.
Complicated or difficult, often multistage ligand syntheses (Buchwald et al., WO0002887), complicated isolation of the product by chromatography.
The removal of the catalyst from the product is often difficult since the products formed bind the transition metals quite effectively, but, on the other hand, very low specification limits have to be observed especially for pharmaceutical fine chemicals (e.g. <10 or <5 ppm). In addition, the customarily used catalyst systems are highly active in various other reactions, such that undesired side reactions can also be catalyzed in subsequent stages.

The present process solves all of these problems and relates to a process for preparing 2-aryl or heteroarylcarbonyl- or -nitrate compounds (III) by cross-coupling enolizable carbonyl compounds, nitrites and analogues thereof (II) with substituted aryl or heteroaryl compounds (I) in the presence of a Brønsted base and of a catalyst or precatalyst comprising a.) a transition metal, a complex, a salt or a compound of this transition metal from the group of {V, Mn, Fe, Co, Ni, Cu, Rh, Pd, Ir, Pt}) and b.) at least one sulfonated phosphine ligand in a solvent or solvent mixture according to Scheme 1.

The process according to the invention is notable for the following advantages:
At very high catalyst loadings, high yields and very high selectivities are achieved.
It utilizes sulfonated ligands which are simple and inexpensive to obtain (ligands which are commercially available by sulfonation or simple to obtain, for example: the 2-hydroxy-2′-dialkyl phosphinobiaryls which are obtainable in a simple and very inexpensive manner according to U.S. Pat. No. 5,789,623 can be converted by simple treatment with sulfuric acid to the corresponding sulfonated ligands. By virtue of the simple obtainability of the corresponding oxaphosphorin chlorides (e.g. 10-chloro-10H-9-oxa-10-phosphaphenanthrene), the reaction is overall a very simple two-stage reaction which proceeds with good yields and is notable for very high flexibility, since a wide variety of different radicals can be introduced in a very simple manner on the phosphorus.)
The catalyst activities achieved by the process according to the invention are very high, since the ligand is present as an anion in the reaction mixture and as a result has particular electronic effects.
Fine tuning of the electronic properties of the inventive ligands is possible by virtue of the possibility of different counterions (metal cations, substituted ammonium salts, etc). Especially in the case of double deprotonatable ligands, for example in the case of sulfonated 2-hydroxy-2′-dialkyl phosphinobiphenyls, it is possible here in a very controlled manner to tailor them to the particular requirements of a certain reaction.
Simple removal of the ligand and metal from the product by aqueous extraction, since, as a result of the very high acidity/polarity of the sulfonated ligands, they preferably reside in the aqueous phase.
The reaction can also be performed in protic solvents, for example substituted alcohols, with an often positive influence on the selectivity/reactivity.
By virtue of the additionally finely adjustable parameters mentioned, the process according to the invention widens the scope of application of the CHC coupling technologies known to date to an exceptional degree.
Exceptional activity of the sulfonated ligands/catalyst systems, and as a result often rapid reactions and short reaction times.

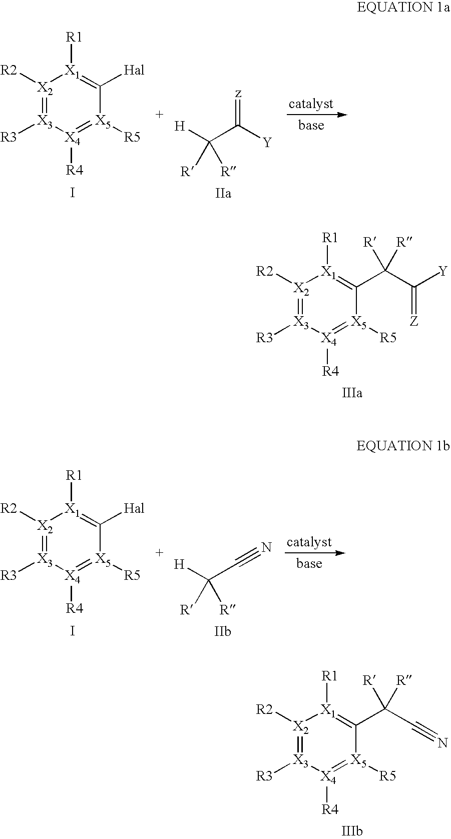

In equation 1a and 1b, Hal is fluorine, chlorine, bromine, iodine, alkoxy or a sulfonate leaving group, for example trifluoromethanesulfonate (triflate), nonafluorotrimethylmethanesulfonate (nonaflate), metlianesulfonate, benzenesulfonate, para-toluenesulfonate, 2-naphthalenesulfonate, 3-nitrobenzenesulfonate, 4-nitrobenzenesulfonate, 4-chlorobenzenesulfonate, 2,4,6-triisopropylbenzenesulfonate.

X₁₋₅ are each independently carbon or nitrogen, or in each case two adjacent XᵢRᵢ bonded via a formal double bond together are 0 (furans), S (thiophenes), NH or NR′ (pyrroles).

Preferred compounds of the formula (I) which can be converted by the process according to the invention are, for example, benzenes, pyridines, pyrimidines, pyrazines, pyridazines, furans, thiophenes, pyrroles, arbitrarily N-substituted pyrroles or naphthalenes, quinolines, indoles, benzofurans, etc.

The R₁₋₅ radicals are each substituents from the group of (hydrogen, methyl, primary, secondary or tertiary, cyclic or acyclic alkyl radicals having from 2 to 20 carbon atoms, in which one or more hydrogen atoms are optionally replaced by fluorine or chlorine or bromine, for example CF₃, substituted cyclic or acyclic alkyl groups, hydroxyl, alkoxy, amino, alkylamino, dialkylamino, arylamino, diarylamino, alkylarylamino, pentaflurorosulfuranyl, phenyl, substituted phenyl, heteroaryl, substituted heteroaryl, thio, alkylthio, arylthio, diarylphosphino, dialkylphosphino, alkylarylphosphino, optionally substituted aminocarbonyl, CO₂, alkyl- or aryloxycarbonyl, hydroxyalkyl, alkoxyalkyl, fluorine or chlorine, nitro, cyano, aryl or alkyl sulfone, aryl- or alkylsulfonyl), or in each case two adjacent R₁₋₅ radicals together may form an aromatic, heteroaromatic or aliphatic fused-on ring Z is O, S, NR′″ (protected imine), NOR′″ (protected oxime), NNR′″R″″ (double-protected hydrazone), or Z, together with Y, is N (nitrile) (equation 1b).

R′, R″, R′″ and R″″ are each independently identical or different radicals from the group of {hydrogen, methyl, linear, branched C₁-C₂₀ alkyl, or cyclic, optionally substituted alkyl, substituted or unsubstituted aryl or heteroaryl, or a functional group not involved in the reaction, for example carbonyl, carboxyl, N-substituted imine or nitrile} or two substituents Rⁱ, together or with an adjacent substituent, form a ring.

Y may be a radical from the group of {hydrogen, methyl, linear, branched C₁-C₂₀-alkyl or cyclic, optionally substituted alkyl, substituted or unsubstituted aryl or heteroaryl, optionally substituted alkoxy, aryloxy, heteroaryloxy, optionally substituted alkylthio, arylthio, heteroarylthio, optionally substituted dialkylamino, di(hetero) arylamino, alkyl (hetero)-arylamino} and may form a ring with R′, R″, R′″ or R″″.

Typical examples of the compound (II) are thus enolizable ketones, aldehydes, N-substituted imines, thioketones, carboxylic esters, thiocarboxylic esters and nitrites.

According to the invention, the catalyst used is a transition metal, preferably on a support, for example palladium on carbon, or a salt, a complex or an organo-metallic compound of this metal. The transition metal is preferably selected from the following group {V, Mn, Fe, Co, Ni, Cu, Rh, Pd, Ir, Pt}, preference being given to using palladium or nickel, with a sulfonated ligand.

The catalyst can be added in finished form or be formed in situ, for example from a precatalyst by reduction or hydrolysis, or from a metal salt and added ligand by complex formation. The catalyst is used in combination with one or more, but at least one, sulfonated phosphorus ligand.

The metal can be used in any oxidation state. According to the invention, it is used in relation to the reactant (I) in amounts of from 0.0001 mol % to 100 mol % preferably between 0.01 and 10 mol %, more preferably between 0.01 and 1 mol %.

According to the invention, sulfonated phosphine ligands which preferably feature the presence of at least one sulfonic acid group or a salt of a sulfonic acid group in the molecule are used.

Preference is given to using ligands of the structure (IV) depicted below

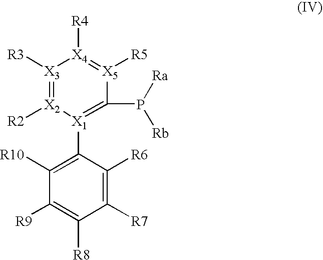

in conjunction with transition metals, preferably palladium or nickel, as the catalyst.

X₁₋₅ are each independently carbon or nitrogen, or in each case two adjacent XᵢRᵢ bonded via a formal double bond, where i=2, 3, 4, 5, together are O (furan), S (thiophene), NH or NRᵢ (pyrrole);

the R₂₋₁₀ radicals correspond in their definition to the R₁₋₅ radicals, where at least one of the radical contains a sulfonic acid or sulfonate group.

R^{a} and R^{b} are each independently identical or different radicals from the group of {hydrogen, methyl, linear, branched or cyclic C₁-C₂₀-alkyl, optionally substituted, phenyl, optionally substituted}, or together form a ring and are a bridging structural element from the group of {optionally substituted alkylene, branched alkylene, cyclic alkylene} or are each independently one or two polycyclic radicals, for example norbornyl or adamantyl.

Particular preference is given here to those derivatives which, as well as at least one sulfonic acid group, also contain a further deprotonatable function in the molecule, for example a free OH group in the sulfonated ring.

In a further preferred embodiment, complexes of a sulfonated secondary phosphine are used in conjunction with a palladacycle as a catalyst of the structure

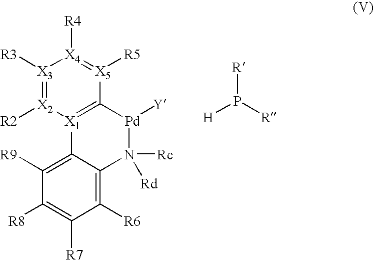

where the symbols X₁₋₅, R₂₋₉, R′ and R″ are each as defined above and Y′ is a radical from the group of {halide, psetidohalide, alkyl carboxylate, trifluoro-acetate, nitrate, nitrite} and
R_{c}, and R^{d} are each independently identical or different substituents from the group of {hydrogen, methyl, primary, secondary or tertiary, optionally substituted C₁-C₂₀-alkyl or aryl}, or together form a ring and stem from the group of {optionally substituted alkylene, oxaalkylene, thiaalkylene, azaalkylene},
and at least one sulfonic acid group or a sulfonate salt is present in the secondary phosphinie.

In a further preferred embodiment, complexes of a tertiary phosphine of the structure

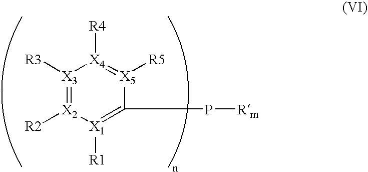

are used, where the symbols X₁₋₅, R₁₋₅ and R′ are each as defined above, where n may be 1, 2 or 3 and m=3-n, and the n aryl or heteroaryl radicals may each independently be of identical or different nature, and the m radicals may likewise each independently be of identical or different nature, where at least one sulfonated aromatic ring is present. Mixtures of different ligands of this class may be used.

Suitable catalysts or precatalysts for the process according to the invention are, for example, complexes of palladium or nickel with sulfonated biaryl-phosphines, some of which are obtainable in a very simple and inexpensive manner (e.g. (VII) and (VIII); for the preparation cf. EP-A-0795559), ox, as representatives of the third type described, the commercially available sulfonated triphenylphosphines (formulae (IX a-c)) TPPTS, TPPDS and TPPMS,

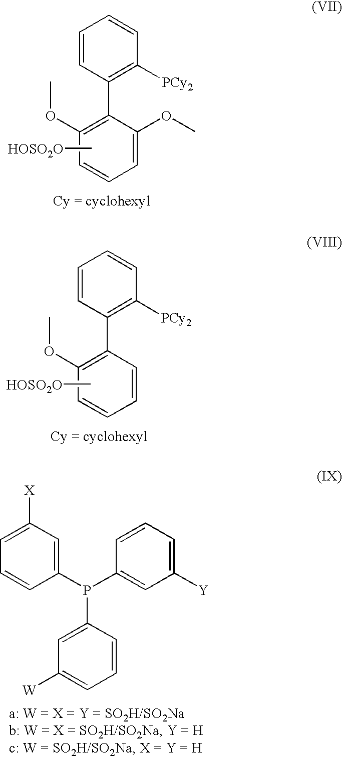

The addition of Brønsted bases to the reaction mixture is necessary in order to achieve acceptable reaction rates. Very suitable bases are, for example, hydroxides, alkoxides and fluorides of the alkali metals and alkaline earth metals, carbonates, hydrogen-carbonates, phosphates, amides and silazides of the alkali metals, and mixtures thereof. Particularly suitable bases are those from the group of {potassium tert-butoxide, sodium tert-butoxide, cesium tert-butoxide, lithium tert-butoxide and the corresponding isopropoxides, potassium hexamethyldisilazide, sodium hexamethyldisilazide, lithium hexamethyldisilazide}.

Typically, at least the amount of base which corresponds to the amount of the compound to be coupled is used; usually from 1.0 to 6 equivalents, preferably from 1.2 to 3 equivalents, of base are used, based on the compound (II).

The reaction is performed in a suitable solvent or a monophasic or polyphasic solvent mixture which has a sufficient dissolution capacity for all reactants involved, and heterogeneous performance is also possible (for example use of almost insoluble bases). Preference is given to performing the reaction in polar, aprotic or protic solvents. Very suitable solvents are dimetlxylformamide (DMF), dimethylacetamide (DMAc), N-methylpyrrolidonie (NMP) dimethyl sulfoxide (DMSO), open-chain and cyclic ethers and diethers, oligo- and polyethers, and substituted mono- or poly-alcohols and optionally substituted aromatics. Particular preference is given to using one solvent or mixtures of a plurality of solvents from the group of {dimethylformamide (DMF), dimethylacetamide (DMAc), N-methylpyrrolidone (NMP), diglyme, substituted glymes, 1,4-dioxane, isopropanol, tert-butanol, 2,2-dimethyl-1-propanol, toluene, xylene).

The reaction can be performed at temperatures in the range from room temperature up to the boiling point of the solvent used at the pressure used. In order to achieve a more rapid reaction, preference is given to performance at elevated temperatures in the range from 0 to 240° C. Particular preference is given to the temperature range from 10 to 200° C., especially from 20 to 150° C.

The concentration of the reactants (I) and (II) can be varied within wide ranges. Appropriately, the reaction is performed in a maximum concentration, though the solubilities of the reactants and reagents in the particular reaction medium have to be considered. Preference is given to performing the reaction in the range between 0.05 and 5 mol/l based on the reactant present in deficiency (depending on the relative costs of the reactants).

The carbonyl derivative or analogue of the formula (II) and aromatic or heteroaromatic reactant (I) may be used in molar ratios of from 10:1 to 1:10; preference is given to ratios of from 3:1 to 1:3 and particular preference to ratios of from 1.2:1 to 1:1.2.

In one of the preferred embodiments, all materials are initially charged and the mixture is heated to reaction temperature with stirring. In a further preferred embodiment which is particularly suitable for use on a large scale, the compound (II) and any further reactants, for example base and catalyst or pre-catalyst, is metered into the reaction mixture during the reaction. Alternatively, it can also be carried out by slow addition of the base under metering control.

The workup is typically effected with a mixture of aromatic hydrocarbons/water with removal of the aqueous phase, which takes up the inorganic constituents and also ligand and transition metal, the product remaining in the organic phase unless acidic functional groups present lead to a different phase behavior. Optionally, ionic liquids can be used to remove the more polar constituents. The product is preferably isolated from the organic phase by precipitation or distillation, for example by concentration or by addition of precipitants. Usually, additional purification or subsequent removal of transition metal or ligand, for example by recrystallization or chromatography, is unnecessary.

The isolated yields for ketones and their derivatives are usually in the range from 60 to 100%, preferably in the range from >70% to 90%, and, for malonates and their derivatives, usually in the range of 50-80%, preferably from >60% to 80%. The selectivities are very high in accordance with the invention; it is usually possible to find conditions under which no further by-products are detectable apart from very small amounts of dehalogenation product.

In particular in the workup and removal of catalyst/ligands, the process according to the invention opens up a very economic method of preparing 2-arylated or -heteroarylated carbonyl compounds, their derivatives and analogues, and also nitrites, proceeding from the corresponding carbonyl compounds or their derivatives and nitrites and the corresponding aryl or heteroaryl halides or aryl or heteroaryl sulfonates, and affords the products generally in very high purities without complicated purification procedures.

The process according to the invention will be illustrated by the examples which follow, without restricting the invention thereto:

### EXAMPLE 1

### Preparation of the ligand 2′-hydroxy-2-di-cyclohexylphosphinobiphenyl-4′-suilfonic acid (HBPNS)

1.099 g (3.0 mmol) of 2-hydroxy-2′-diphenylphosphino-biphenyl were precooled in an ice bath under a protective gas atmosphere. Subsequently, 2.0 ml of concentrated sulfuric acid were metered in slowly from a syringe. After warming up to room temperature, the suspension formed was stirred for a further approx. 2 hours until all solid had dissolved. A homogeneous, viscous and slightly brownish suspension was obtained.

The reaction mixture was cooled again in an ice bath and then quenched with ice. Concentrated sodium hydroxide solution was used to dissolve the precipitate formed completely. After dilution with 75 ml of water and acidification with 1 N sulfuric acid, the precipitate was filtered off and washed with water until the effluent washwater exhibited a neutral pH. The white filtercake was washed once more with methanol and dried under reduced pressure. 1.093 g (2.45 mmol, 82%) of 2-hydroxy-2-diplienylphosplhinobiplhenyl-5-sulfonic acid were obtained as white crystals.

### EXAMPLE 2

### Coupling of 4-bromobenzonitrile with acetophenone to give 4-(2-oxo-2-phenylethyl)benzonitrile

182 mg of 4-bromobenzonitrile (1 mmol) and 120 mg of acetophenone (1 mmol) were dissolved in 5 ml of N,N-dimethylformamide under protective gas and admixed with 192 mg of sodium tert-butoxide (2 mmol). The mixture was left to stir for 15 min, and then 17.9 mg (4 mol %) of the HBPNS ligand and 9.0 mg of palladium(II) acetate (4 mol %) were added, and the mixture was heated to 80° C. for 14.5 h. For workup, 5 ml of water and 10 ml of toluene were added, the mixture was shaken, and the lower water phase was discharged and washed once again with 5 ml of water to remove residual dimethylformamide. The solvent was removed on a rotary evaporator under reduced pressure. 175 mg of the product were obtained (0.79 mmol, 79%).

### EXAMPLE 3

### Coupling of 4-bromobenzonitrile with cyclo-hexanone to give 4-(2-oxocyclohexyl)benzonitrile

182 mg of 4-bromobenzonitrile (1 mmol) and 98 mg of cyclohexanone (1 mmol) were dissolved in 5 ml of N,N-dimethylformamide under protective gas and admixed with 192 mg of sodium tert-butoxide (2 mmol). The mixture was left to stir for 15 min and then 17.9 mg (4 mol %) of the HBPNS ligand and 9.0 mg of palladium(II) acetate (4 mol %) were added, and the mixture was heated to 80° C. for 14.5 h. For workup, 5 ml of water and 10 ml of toluene were added, the mixture was shaken, and the lower water phase was discharged and washed once again with 5 ml of water to remove residual dimethylformamide. The solvent was removed on a rotary evaporator under reduced pressure. After flash chromatography (10:1 cyclohexane/ethyl acetate), 111.6 mg of the product were obtained (0.56 mmol, 56%).

### EXAMPLE 4

### Coupling of 4-bromoanisole with acetophenone to give 2-(4-methoxyphenyl)-1-phenylethanone

187 mg of 4-bromoanisole (1 mmol) and 120 mg of acetophenone (1 mmol) were dissolved in 5 ml of N,N-dimethylformamide under protective gas and admixed with 192 mg of sodium tert-butoxide (2 mmol). The mixture was left to stir for 15 min, and then 17.9 mg (4 mol %) of the HBPNS ligand and 9.0 mg of palladium(II) acetate (4 mol %) were added, and the mixture was heated to 80° C. for 14.5 h. For workup, 5 ml of water and 10 ml of toluene were added, the mixture was shaken, and the lower water phase was discharged and washed once again with 5 ml of water to remove residual dimethylformamide. The solvent was removed on a rotary evaporator under reduced pressure. 185 mg of the product were obtained (0.82 mmol, 82%).

### EXAMPLE 5

### Coupling of 4-bromoanisole with cyclo-hexanone to give 2-(4-methoxyphenyl)cyclohexanone

187 mg of 4-bromoanisole (1 mmol) and 98 mg of cyclohexanone (1 mmol) were dissolved in 5 ml of N,N-dimethylformamide under protective gas and admixed with 192 mg of sodium tert-butoxide (2 mmol). The mixture was left to stir for 15 min, and then 17.9 mg (4 mol %) of the HBPNS ligand and 9.0 mg of palladium(II) acetate (4 mol %) were added, and the mixture was heated to 80° C. for 20 h. For workup, 5 ml of water and 10 ml of toluene were added, the mixture was shaken, and the lower water phase was discharged and washed once again with 5 ml of water to remove residual dimethylformamide. The solvent was removed on a rotary evaporator under reduced pressure. After flash chromatography (10:1 cyclohexane/ethylacetate), 146 mg of the product were obtained (0.71 mmol, 71%).

### EXAMPLE 6

### Coupling of 4-chlorobromobenzene with diethyl malonate to give diethyl 2-(4-chlorophenyl) malonate

191.5 mg of 4-chlorobromobenzene (1 mmol) and 160 mg of diethyl malonate (1 mmol) were dissolved in 5 ml of N,N-dimethylformamide under protective gas, admixed with 652 mg of cesium carbonate (2 mmol) and stirred for 1 h. 17.9 mg (4 mol %) of the HBPNS ligand and 9.0 mg of palladium(II) acetate (4 mol %) were then added, and the mixture was heated to 80° C. for 24 h.

For workup, 5 ml of water and 10 ml of toluene were added, the mixture was shaken, and the lower water phase was discharged and washed once again with 5 ml of water to remove residual dimethylformamide. After removal of the toluene on a rotary evaporator, 230 mg (0.85 mmol, 85%) of the product were obtained.

### EXAMPLE 7

### Coupling of 4-chlorobromobenzene with ethyl cyanoacetate to give ethyl 4-chlorophenylcyanoacetate

191.5 mg of 4-chlorobromobenzene (1 mmol) and 113 mg of ethyl cyanoacetate (1 mmol) were dissolved in 5 ml of N,N-dimethylformamide under protective gas, admixed with 652 mg of cesium carbonate (2 mmol) and stirred for 1 h. 17.9 mg (4 mol %) of the HBPNS ligand and 9.0 mg of palladium(II) acetate (4 mmol) were then added, and the mixture was heated to 80° C. for 24 h. For workup, 5 ml of water and 10 ml of toluene were added, the mixture was shaken, and the lower water phase was discharged and washed once again with 5 ml of water to remove residual dimethylformamide. After removal of the toluene on a rotary evaporator, 166 mg (0.74 mmol, 74%) of the product were obtained.

### EXAMPLE 8

### Coupling of 4-chlorobromobenzene with malononitrile to give 1-chloro-4-dicyanomethylbenzene

191.5 mg of 4-chlorobromobenzene (1 mmol) and 66 mg of malononitrile (1 mmol) were dissolved in 5 ml of N,N-dimethylformamide under protective gas, admixed with 343 mg of barium hydroxide (2 mmol) and stirred for 1 h. 17.9 mg (4 mole) of the HBPPS ligand and 9.0 mg of palladium(II) acetate (4 mol %) were then added, and the mixture was heated to 80° C. for 24 h. For workup, 5 ml of water and 10 ml of toluene were added, the mixture was shaken, and the lower water phase was discharged and washed once again with 5 ml of water to remove residual dimethylformamide. After removal of the toluene on a rotary evaporator, 149 mg (0.85 mmol, 85%) of the product were obtained.

### EXAMPLE 9

### Coupling of Ethyl Phenylacetate with 4-bromotoluene to give ethyl phenyl-p-tolylacetate

164 mg of ethyl phenylacetate (1 mmol) and 171 mg of 4-bromiotoluene (1 mmol) were admixed with 224 mg of potassium tert-butoxide (2 mmol) at room temperature under protective gas, and the mixture was stirred for 30 min. 17.9 mg (4 mol %) of the HBPNS ligand and 9.0 mg of palladium(II) acetate (4 mol %) were then added, and the mixture was heated to 80° C. for 3.5 h.

For workup, 5 ml of water and 10 ml of toluene were added, the mixture was shaken, and the lower water phase was discharged and washed once again with 5 ml of water to remove residual dimethylformamide. After removal of the toluene on a rotary evaporator and flash chromatography (10:1 cyclohexane/ethyl acetate), 176 mg (0.69 mmol, 69%) of the product were obtained.

### EXAMPLE 10

### Coupling of 4-bromobenzonitrile with octanal to give 4-(1-formylheptyl)benzonitrile

182 mg of 4-bromobenzonitrile (1 mmol) and 128 mg of octanal (1 mmol) were dissolved in 5 ml of N,N-dimethylformamide under protective gas and admixed with 192 mg of sodium tert-butoxide (2 mmol). The mixture was left to stir for 15 min, and then 17.9 mg (4 mol %) of the HBPNS ligand and 9.0 mg of palladium(II) acetate (4 mol %) were added, and the mixture was heated to 80° C. for 14.5 h. For workup, S ml of water and 10 ml of toluene were added, the mixture was shaken, and the lower water phase was discharged and washed once again with 5 ml of water to remove residual dimethylformamide. The solvent was removed on a rotary evaporator under reduced pressure. 136 mg of the product were obtained (0.57 mmol, 57%)+

### EXAMPLE 11

### Coupling of 4-bromobenzonitrile with phenylacetaldehyde to give 4-(2-oxo-1-phenylethyl)-benzonitrile

182 mg of 4-bromobenzonitrile (1 mmol) and 120 mg of phenylacetaldehyde (1 mmol) were dissolved in 5 ml of N,N-dimethylformamide under protective gas and admixed with 192 mg of sodium tert-butoxide (2 mmol). The mixture was left to stir for 15 min, and then 17.9 mg (4 mol %) of the HBPNS ligand and 9.0 mg of palladium(II) acetate (4 mol %) were added, and the mixture was heated to 80° C. for 14.5 h. For workup, 5 ml of water and 10 ml of toluene were added, the mixture was shaken, and the lower water phase was discharged and washed once again with 5 ml of water to remove residual dimethylformamide. The solvent was removed on a rotary evaporator under reduced pressure. 150 mg of the product were obtained (0.65 mmol, 65%).

### EXAMPLE 12

### Coupling of 4-bromobenzotrifluoride with phenylacetonitrile to give phenyl (4-trifluoromethyl)-acetonitrile

117 mg of phenylacetonitrile (1 mmol) and 225 mg of 4-bromobenzotrifluoride (1 mmol) were admixed with 224 mg of potassium tert-butoxide (2 mmol) at room temperature under protective gas, and the mixture was stirred for 30 min. 17.9 mg (4 mol-0) of the HBPNS ligand and 9.0 mg of palladium(II) acetate (4 mol %) were then added, and the mixture was heated to 80° C. for 3.5 h.

For workup, 5 ml of water and 10 ml of toluene were added, the mixture was shaken, and the lower water phase was discharged and washed once again with 5 ml of water to remove residual dimethylformamide. After removal of the toluene on a rotary evaporator and flash chromatography (10:1 cyclohexane/ethyl acetate), 165 mg (0.76 mmol, 76%) of the product were obtained.

### EXAMPLE 13

### Coupling of 4-bromobenzotrifluoride with isobutyronitrile to give 2-methyl-2-(4-trifluoromethyl-phenyl)propionitrile

69 mg of isobutyronitrile (1 mmol) and 225 mg of 4-bromobenzotrifluoride (1 mmol) were admixed with 334 mg of lithium hexamethyldisilazide (2 mmol) at room temperature under protective gas, and the mixture was stirred for 30 min. 17.9 mg (4 mol %) of the HBPNS ligand and 9.0 mg of palladium(II) acetate (4 mol %) were then added, and the mixture was heated to 80° C. for 10 h.

For workup, 5 ml of water and 10 ml of toluene were added, the mixture was shaken, and the lower water phase was discharged and washed once again with 5 ml of water to remove residual dimethylformamide. After removal of the toluene on a rotary evaporator and flash chromatography (10:1 cyclohexane/ethyl acetate), 101 mg (0.55 mmol, 55%) of the product were obtained.

### EXAMPLE 14

### Coupling of N-diphenylmethyleneglycine ethyl ester with bromobenzene to give 2-N-diphenyl-methylene-2-aminophenylacetic acid

267 mg of N-diphenylmethyleneglycine ethyl ester (1 mmol) and 157 mg of bromobenzene (1 mmol) were admixed with 224 mg of potassium tert-butoxide (2 mmol) at room temperature under protective gas, and the mixture was stirred for 30 min. 17.9 mg (4 mol %) of the HBPNS ligand and 9.0 mg of palladium(II) acetate (4 mol %) were then added, and the mixture was heated to 80° C. for 24 h.

For workup, 5 ml of water and 10 ml of toluene were added, the mixture was shaken, and the lower water phase was discharged and washed once again with 5 ml of water to remove residual dimethylformamide. After removal of the toluene on a rotary evaporator and flash chromatography (10:1 cyclohexane/ethyl acetate), 282 mg (0.82 mmol, 82%) of the product were obtained.

1. A process for preparing compounds of the formula (III) comprising cross-coupling enolizable carbonyl compounds) nitriles or analogues thereof of the formula (II) with substituted aryl or heteroaryl compounds of the formula (I) in the presence of a Brønsted base and of a catalyst or precatalyst comprising
a.) a transition metal, a complex, a salt or a compound of said transition metal from the group of V, Mn, Fe, Co, Ni, Rh, Pd, Ir, Pt, and
b.) at least one sulfonated phosphine ligand
in a solvent or solvent mixture according to Reaction Scheme 1 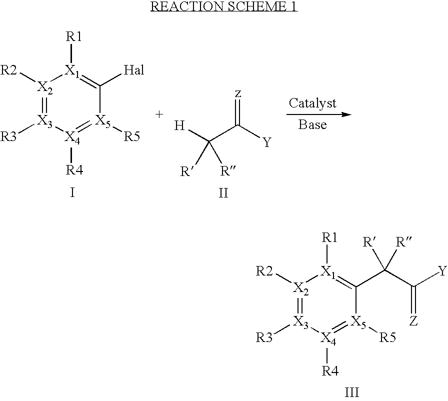
where Hal is fluorine, chlorine, bromine, iodine, alkoxy or a sulfonate group;
X₁₋₅ are each independently carbon or nitrogen or in each case two adjacent XᵢRᵢ bonded via a formal double bond together are O, S, NH or NR′;
the R₁₋₅ radicals are each substituents from the group of hydrogen, methyl, primary, secondary or tertiary, cyclic or acyclic alkyl radicals having from 2 to 20 carbon atoms, in which one or more hydrogen atoms are optionally replaced by fluorine or chlorine or bromine, cyclic or acyclic alkyl groups, hydroxyl, alkoxy, amino, alkylamino, dialkylamino alkylamino, arylaminio, diarylamino, alkyl arylaminmo, pentafluorosulfuranyl, phenyl, substituted phenyl, heteroaryl, substituted heteroaryl, thio, alkylthio, arylthio, diarylphosphino, dialkylphosphino, alkylarylphosphino, aminocarbonyl, CO₂—, alkyl- or aryloxycarbonyl, hydroxyalkyl, alkoxyalkyl, fluorine or chlorine, nitro, cyano, aryl or alkyl sulfone, aryl- or alkylsulfanyl or in each case two adjacent R₁₋₅ radicals together form an aromatic, heteroaromatic or aliphatic fused-on ring,
Z is O, S, NR′″, NOR′″, NNR′″R″″, or Z together with Y forms a CN group,
R′, R″, R′″ and R″″ are each identical or different radicals from the group of hydrogen, methyl, linear, branched C₁-C₂₀ alkyl, or cyclic, optionally substituted alkyl, substituted or unsubstituted aryl or heteroaryl, or a functional group not involved in the reaction, or two substituents Ri, together or with an adjacent substituent, form a ring,
Y is a radical from the group of hydrogen, methyl, linear, branched C₁-C₂₀-alkyl or cyclic, optionally substituted alkyl, substituted or unsubstituted aryl or heteroaryl, optionally substituted alkoxy, aryloxy, heteroaryloxy, optionally substituted alkylthio, arylthio, heteroarylthio, optionally substituted dialkyl-amino, di(hetero)arylamino, alkyl(hetero) arylamino and may form a ring with R′, R″, R′″ or R″″.

2. The process as claimed in claim 1, wherein sulfonated phosphine ligands which contain at least one sulfonic acid group or a metal sulfonate are used.

3. The process as claimed in claim 1, wherein the Brønsted base used is an alkoxide or amide of the alkali metals or alkaline earth metals, or an alkali metal carbonate or phosphate or silazide, or mixtures of these compounds.

4. The process as claim in claim 1, wherein from 1.0 to 3 equivalents of base are used based on the aryl halide or heteroaryl halide or aryl sulfonate or heteroaryl sulfonate.

5. The process as claimed in claim 1, wherein the solvents used are hydrocarbons, halogenated hydrocarbons, open-chain and cyclic ethers and diethers, oligoethers and polyethers, tertiary amines, dimethyl sulfoxide, N-methylpyrrolidone, dimethylformamide dimethylacetamide and substituted mono- or polyalcohols and optionally substituted aromatics or a mixture of a plurality of these solvents.

6. The process as claimed in claim 1, wherein the cross-coupling reaction is performed at a temperature in the range from 0 to 240° C.

7. The process as claimed in claim 1, wherein the catalyst is used in relation to the reactant (1) in amounts of from 0.001 mmol % to 100 mol %.

8. The process as claimed in claim 1, wherein a phosphinic ligand Of the stricture 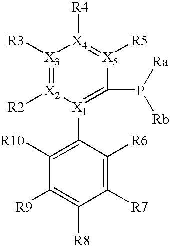
is used, where
X₁₋₅ are each independently carbon or nitrogen, or in each case two adjacent XᵢRᵢ are bonded via a formal double bond, where i=2, 3, 4, 5, together are O, S, NH or NRᵢ;
the R₂₋₁₀ radicals correspond in their definition to the R₁₋₅ radicals in claim 1, where at least one radical contains a sulfonic acid or sulfonate group;
Ra and Rb are each independently identical or different radicals from the group of hydrogen, methyl, linear, branched or cyclic C₁-C₂₀-alkyl, phenyl, or together form a ring and are a bridging structural element from the group of alkylene, branched alkylene, cyclic alkylene or are each independently one or two polycyclic radicals.

9. The process as claimed in claim 1, wherein the phosphine ligand and catalyst used is a complex of a sulfonated secondary phosphine in conjunction with a palladacycle of the formula (V) 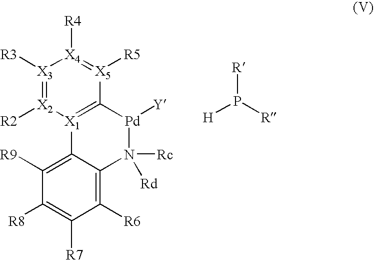
where the symbols X₁₋₅, R₂₋₉, R′ and R″ are cacti as defined in claim 1 and Y′ is a radical from the group of halide, pseudohalide, alkyl carboxylate, trifluoroacetate nitrate, nitrite and
R_{c} and R_{d} are each independently identical or different substituents from the group of hydrogen, methyl, primary, secondary or tertiary, optionally substituted C₁-C₂₀-alkyl or aryl, or together form a ring and stern from the group of optionally substituted alkylene, oxaalkylene, thiaalkylene, azaalkylene, and at least one sulfonic, acid group or a sulfonate salt is present in the secondary phosphine.

10. The process as claimed in claim 1, wherein the phosphine ligand used is a complex of a sulfonated tertiary phosphine of the formula (VI) 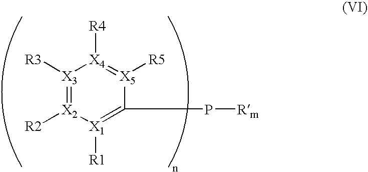
where the symbols X₁₋₅, R₁₋₅ and R′ are each as defined in claim 1, where n may be 1, 2 or 3 and m=3-n, and the n aryl or heteroaryl radicals and the m radicals may each independently be the same or different, and mixtures of different ligands of this class may be used.

11. The process as claimed in claim 1, wherein R′, R″, R′″ and R″″ are each identical or different radicals from the group of hydrogen, methyl, linear, branched C₁-C₂₀ alkyl, or cyclic, optionally substituted alkyl, substituted or unsubstituted aryl or heteroaryl, or carbonyl, carboxyl, N-substituted imine or nitrile or two substituents Ri, together or with an adjacent substituent, form a ring.
